(19) United States
(12) Patent Application Publication
(10) Pub. No.: US 2019/0176155 A1
(43) Pub. Date: Jun. 13, 2019

(54) DETECTING APPARATUS FOR DNA OR RNA, KIT COMPRISING SAME, AND SENSING METHOD FOR DNA OR RNA

(71) Applicant: Electronics and Telecommunications Research Institute, Daejeon (KR)

(72) Inventors: Hyeon-Bong PYO, Daejeon (KR); Jeong Won PARK, Daejeon (KR)

(21) Appl. No.: 16/217,566

(22) Filed: Dec. 12, 2018

(30) Foreign Application Priority Data
Dec. 12, 2017 (KR) 10-2017-0170724
Nov. 21, 2018 (KR) 10-2018-0144900

(51) Int. Cl. B01L 7/00; C12Q 1/6809

(52) U.S. Cl. CPC B01L 7/52; B01L 2300/0636; C12Q 1/6809

(57) ABSTRACT

A detecting apparatus for at least one of DNA and RNA includes: a substrate; at least one chamber formed on the surface of the substrate; a plurality of nanostructures fixed on the internal surface of the at least one chamber; at least one light source configured to supply incident light on the at least one chamber; and a light-receiving unit configured to sense an optical signal from the nanostructures.

### CROSS-REFERENCE TO RELATED APPLICATION

This application claims priority to and the benefit of Korean Patent Application Nos. 10-2017-0170724 and 10-2018-0144900 filed in the Korean Intellectual Property Office on Dec. 12, 2017, and Nov. 21, 2018, respectively, the entire contents of which are incorporated herein by reference.

### BACKGROUND OF THE INVENTION

### (a) Field of the Invention

A detecting apparatus for DNA or RNA, a kit including the detecting apparatus, and a sensing method for DNA or RNA are disclosed.

### (b) Description of the Related Art

In order to elute intracellular DNA, a mechanical force using a vortex mixer or the like, or a method of treating a chemical substance such as lysozyme, EDTA (ethylenediaminetetraacetate) or SDS (sodium dodecyl sulfate) has been used.

DNA extracted from cells is amplified by polymerase chain reaction (PCR) and analyzed. The polymerase chain reaction (PCR) is a method for amplifying specific DNA fragments and is a core technology in the fields of modern molecular biology, biochemistry, genetic engineering, and the like.

In a typical polymerase chain reaction, a DNA double strand is denatured into a single strand at about 95° C., the temperature is lowered to anneal a primer having a base sequence complementary to the substrate of the DNA single strand, and the temperature is increased again so that a DNA polymerase may polymerize dNTP complementary to the DNA strand from the 3′-OH end of the primer to which the DNA single strand is bound to synthesize the DNA double strand. The DNA repeats a cycle of denaturation-annealing-polymerization in a predetermined number of times, and the DNA is amplified using the synthesized DNA double helix as a substrate of the next cycle.

Recently, amplified DNA is quantified by the real-time PCR method which may measure the amount of amplified DNA quantitatively, in each cycle in real time, by adding a fluorophore as a reporter in the PCR reaction. The real-time PCR may be measured directly by measuring an amount of amplified DNA or by a TaqMan method of quantifying the fluorescence generated by using a dye that binds to DNA and a probe DNA depending on experimental methods.

In order to carry out a series of processes of dissolving cells to extract and amplify DNA, complex and precise large-scale equipment such as various physicochemical means and a thermo-cycling apparatus (thermo-cycler) is required, and quantitative measurement of the amplified DNA is required, and in addition, independent sensing means are separately required.

### SUMMARY OF THE INVENTION

An embodiment provides at least one detecting apparatus of DNA and RNA capable of denaturating, amplifying, and detecting one or more of DNA and RNA on a single substrate.

Another embodiment provides a kit including the apparatus.

Yet another embodiment provides a method of sensing at least one of DNA and RNA using the apparatus.

An embodiment provides a detecting apparatus for at least one of DNA and RNA, including: a substrate; at least one chamber formed on the substrate; a plurality of nanostructures fixed on the internal surface of the at least one chamber; at least one light source configured to supply incident light on the at least one chamber; and a light-receiving unit configured to sense an optical signal from the nanostructures.

The at least one chamber may include at least one first heating chamber and at least one sensing chamber.

The at least one chamber may further include at least one sample chamber, at least one fluid channel connecting the at least one sample chamber with the at least one first heating chamber, and at least one fluid channel connecting the at least one first heating chamber with the at least one sensing chamber.

The at least one chamber may further include at least one reagent chamber, and at least one fluid channel connecting the at least one reagent chamber with the at least one first heating chamber.

The substrate may be transparent glass or a polymer having a refractive index of about 1.3 to about 1.9.

The glass may include one of SiO₂, BK7, SF10, SF11, N-LASF46A, and a combination thereof, and the polymer may include a polystyrene-based polymer, a polymethylmethacrylate polymer, a polycarbonate-based polymer, a cyclic olefin copolymer, or a combination thereof.

The nanostructures may be spherically-shaped metal nanoparticles or nanoparticles having a core-shell shape where the core is a dielectric and the shell is a metal, or a combination thereof.

In the spherically-shaped metal nanoparticles, the metal may be gold (Au), silver (Ag), copper (Cu), aluminum (Al), or a combination thereof.

In the nanoparticle having a core-shell shape where the core is a dielectric and the shell is a metal, the dielectric may include SiO₂, BK7, SF10, SF11, N-LASF46A, a polystyrene-based polymer, a polymethylmethacrylate polymer, a polycarbonate-based polymer, a cyclic olefin copolymer, or a combination thereof, and the metal may include gold (Au), silver (Ag), copper (Cu), aluminum (Al), or a combination thereof.

A size of the nanostructures may be about 1 nm to about 1000 nm.

An internal surface of the at least one first heating chamber may have a reflective coating.

The at least one sensing chamber may include at least one thermo-cooler.

The at least one light source may irradiate light in a wavelength range of about 10 nm to about 10 μm.

The at least one light source may independently irradiate light having a wavelength range configured to increase a temperature of the surrounding medium by inducing a photothermal phenomenon of the nanostructures,

light having a wavelength range configured to perform plasmon resonance absorption of the nanostructures, maximization of scattering efficiency, or both, or

light having a wavelength range configured to perform plasmon resonance absorption of the nanostructures by inducing a photothermal phenomenon of the nanostructures and to maximize scattering efficiency, or both, while increasing the temperature of the surrounding medium.

The at least one light source may independently emit monochromatic light or polychromatic light.

The detecting apparatus may further include at least one of a polarizer, a color filter, or a combination thereof between the substrate and the at least one light source.

One or more light receiving units may be present on the same surface as the at least one light source with respect to the substrate, or may be present on opposite surfaces of the at least one light source in the center of the substrate.

The detecting apparatus for at least one of DNA and RNA may further include a control unit configured to control a wavelength of incident light on the at least one chamber and an on/off cycle of the at least one light source.

According to another embodiment, a kit includes the detecting apparatus for at least one of DNA and RNA according to the embodiment, and a reagent to react with a sample including at least one of DNA and RNA.

According to another embodiment, a sensing method of at least one of DNA and RNA includes: adding a sample including one or more of DNA and RNA to be analyzed, a polymerase, and a base to at least one chamber formed on a substrate and having a plurality of nanostructures fixed on an inner surface thereof; irradiating the chamber with light to adjust the temperature of the sample and to perform a polymerization reaction so that at least one of DNA and RNA including a specific sequence in at least one of the DNA and RNA is amplified; and irradiating the chamber in which the polymerization reaction is completed with light to sense an absorbed or scattered optical signal.

The apparatus according to an embodiment generates a photothermal phenomenon of nanostructures and/or a plasmon resonance phenomenon by controlling a size and a material of nanostructures and a wavelength of incident light, so as to provide an all-optical detecting technique for at least one of DNA and RNA in which the at least one of DNA and RNA may be denatured, amplified, and sensed (for example, quantitatively sensed) on a single substrate.

### BRIEF DESCRIPTION OF THE DRAWINGS

FIG. 1 is a schematic top plan view showing a substrate of a detecting apparatus for at least one of DNA and RNA according to an embodiment.

FIG. 2 is a cross-sectional view of a spherically-shaped metal nanoparticle according to an embodiment.

FIG. 3 is a schematic top plan view showing that at least one of the spherically-shaped metal nanoparticles is fixed on an internal surface of a chamber of an apparatus according to an embodiment.

FIG. 4 is a cross-sectional view of a core-shell-shaped nanoparticle according to an embodiment.

FIG. 5 is a schematic top plan view showing that at least one of the core-shell-shaped nanoparticles is fixed on an internal surface of a chamber of an apparatus according to an embodiment.

FIG. 6 is a schematic top plan view of a detecting apparatus including a plurality of chambers formed on a substrate according to an embodiment.

FIG. 7 is a schematic top plan view of a detecting apparatus including a plurality of chambers and at least one fluid channel formed on a substrate according to an embodiment.

FIG. 8 is a schematic cross-sectional side view showing a detecting apparatus according to an embodiment further including a light source and a light-receiving unit disposed above the same surface as the light source, which are not shown in FIG. 7, in addition to the cross-sectional view of the substrate cut along with a line A-A′.

FIG. 9 is a schematic cross-sectional side view showing a detecting apparatus according to an embodiment further including a light source and a light-receiving unit disposed above a surface opposite to the light source leaving a substrate therebetween, which are not shown in FIG. 7, in addition to the cross-sectional view of the substrate cut along with a line A-A′.

FIG. 10 is a graph showing plasmon resonance absorption efficiency (σ_{abs}) of a spherically-shaped gold nanoparticle in water, which is changed depending upon a radius of a spherically-shaped gold nanoparticle.

FIG. 11 is a graph showing a water temperature change (ΔT) according to plasmon resonance absorption of a spherically-shaped gold nanoparticle in water, which is changed depending upon a radius of a spherically-shaped gold nanoparticle.

FIG. 12 shows the results of theoretically calculating plasmon resonance absorption efficiency (σ_{abs}) of a core-shell-shaped nanoparticle in water, which is changed depending upon a radius of the silica core, in the nanoparticle having a core-shell shape where the core is silica and the shell is gold (Au) having a thickness of 1 nm.

FIG. 13 shows the results of theoretically calculating plasmon resonance absorption efficiency (σ_{abs}) of a core-shell-shaped nanoparticle in water, which is changed depending upon a thickness of the shell, in the nanoparticle having a core-shell shape where the core is silica having a radius of 15 nm (a) and the shell is gold (Au).

FIG. 14 schematically illustrates a method of changing a temperature by adjusting an operation time of a light source and a thermo-cooler according to time, in order to amplify and quantitatively sense at least one of DNA and RNA in at least one chamber.

### DETAILED DESCRIPTION OF THE EMBODIMENTS

Advantages and characteristics of this disclosure, and a method for achieving the same, will become evident referring to the following embodiments together with the drawings attached hereto. The embodiments may, however, be embodied in many different forms, and should not be construed as being limited to the embodiments set forth herein. If not defined otherwise, all terms (including technical and scientific terms) in the disclosure may be defined as commonly understood by one skilled in the art. Like reference numerals designate like elements throughout the disclosure.

The terminology used herein is for the purpose of illustrating embodiments and is not intended to be limiting of the disclosure. In the present disclosure, the singular includes the plural unless mentioned otherwise. The terms “comprises” and/or “comprising” used in the disclosure do not exclude the presence or addition of one or more other elements or steps in addition to the stated elements or steps.

In addition, the embodiments described herein will be described with reference to cross-sectional views, side sectional views, and/or plan views, which are ideal illustrations of the present disclosure. In the drawings, the thicknesses of the regions are exaggerated for effective description of the technical disclosure. Thus, the shapes of the illustrations may be modified by manufacturing techniques and/or permissible tolerances. Accordingly, the embodiments of the present disclosure are not limited to the specific shapes shown, but also include changes in the shapes that are produced according to the manufacturing processes. For example, the etching regions shown at perpendicular angles may be in a shape having a predetermined curvature. Thus, the regions illustrated in the drawings have schematic attributes, and the shapes of the regions illustrated in the drawings are intended to illustrate specific types of regions of the elements and are not intended to limit the scope of the disclosure.

When a section of a plate, a substrate, a chamber, a thermo-cooler, a layer, a film, a region, or the like is referred to as being “on” another element, it can be directly on the other element or intervening elements may also be present. In contrast, when an element is referred to as being “directly on” another element, there are no intervening elements present.

An apparatus according to an embodiment includes one substrate and at least one chamber formed on the substrate, and includes a plurality of nanostructures fixed on the internal surface of the chamber. Using the apparatus according to the embodiment, it may detect and/or quantify a variety of samples, and for example, it may amplify and sense DNA and/or RNA.

Specifically, a detecting apparatus according to an embodiment generates a photothermal phenomenon of the nanostructures and/or a plasmon resonance phenomenon by adjusting a size and a material of nanostructures fixed on the internal surface of the chamber, a wavelength of light incident on the chamber, or an on/off cycle of a light source irradiating light to the chamber, so as to all-optically perform the amplification and the sensing of DNA and/or RNA on a single substrate. In an embodiment, the apparatus may elute DNA and/or RNA from cells as well as amplify and detect DNA and/or RNA in at least one chamber. Furthermore, the sensing may be quantitative sensing.

A DNA and/or RNA detecting apparatus according to the embodiment, a kit including the detecting apparatus, and a method of sensing at least one of DNA and RNA using the detecting apparatus will now be described with references to drawings.

FIG. 1 is a schematic top plan view showing a substrate of an apparatus according to an embodiment.

Referring to FIG. 1, the apparatus according to an embodiment includes a substrate 100 and at least one chamber 110 formed on the substrate 100. Although FIG. 1 shows an apparatus in which only one chamber 110 is formed on one substrate 100 according to an embodiment, a plurality of chambers may be formed on one substrate 100.

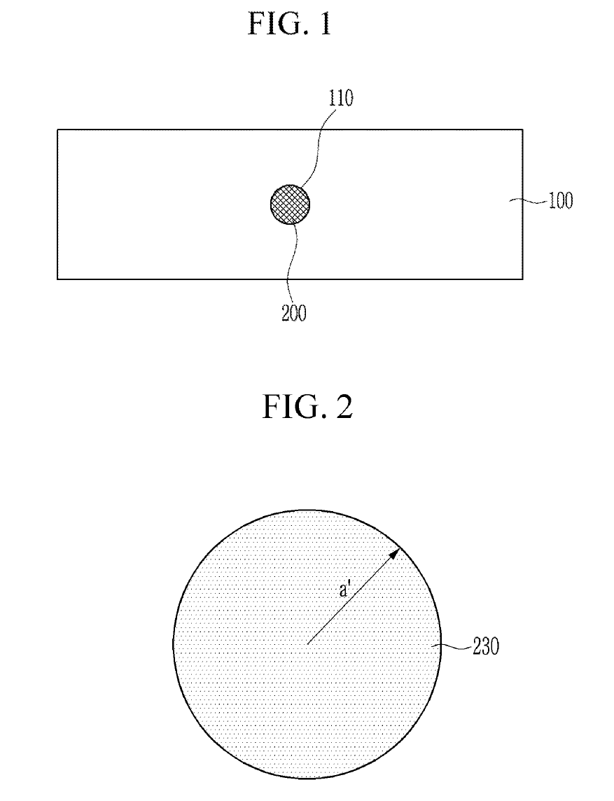

As shown in FIG. 1, the detecting apparatus according to an embodiment may include only one chamber 110, and a plurality of nanostructures 200 may be fixed on the internal surface of one chamber 110 as described later in the description of FIGS. 2 to 5. A sample to be analyzed, for example, DNA and/or RNA, is put into the chamber fixed with a plurality of nanostructures 200, and light is irradiated to the chamber from a light source which is not shown in the drawing. In this case, the nanostructures 200 fixed on the internal surface of the chamber may generate a photothermal reaction by a surface plasmon resonance phenomenon by the light, and the temperature of the surrounding medium of nanostructures 200 may be increased. When the temperature is increased as above, DNA and/or RNA in the sample is denatured to a single strand; a primer, a polymerase, dNTP, and the like for polymerizing DNA and/or RNA are added thereto; and then a wavelength of light incident on one chamber 110, an on/off cycle of the light source, and an on/off cycle of a thermo-cooler are adjusted to change the temperature, so that the DNA and/or RNA may undergo a polymerization reaction. DNA and/or RNA having a specific sequence is amplified by repeating the denaturation and the polymerization reaction of the DNA and/or RNA as above, and one chamber 110 is irradiated with light having a same or different wavelength from the light from the light source which is not shown in the drawing, so intensity of the optical signal emitted or scattered by plasmon resonance absorption of the nanostructures 200 fixed on one chamber 110 or a scattering phenomenon may be sensed to detect DNA and/or RNA having a specific sequence. The sensing may be performed in a light-receiving unit which is not shown, and the sensing may be quantitative sensing.

The substrate 100 may be a flat plate. The substrate 100 may be transparent glass or a polymer having a high refractive index of about 1.3 to about 1.9, 1.4 to 1.8, or 1.4 to 1.7. The glass may be hard glass and the polymer may be bendable or deformable. The glass may include one of a silicate glass (SiO₂), borosilicate glass, BK7 (made by Schott AG), SF10 (made by Schott AG), SF11 (made by Schott AG), N-LASF46A (made by Schott AG), and a combination thereof, and the polymer having a high refractive index may include a polystyrene-based polymer, a polymethylmethacrylate polymer, a polycarbonate-based polymer, a cyclic olefin copolymer, or a combination thereof.

The at least one chamber may support a sample, a reagent, or a combination thereof.

The sample may include at least one of DNA and RNA, and the sample including at least one of DNA and RNA may include cells, a cell suspension, nuclei of cells, extracted DNA, or extracted RNA. The extracted DNA or the extracted RNA may be any DNA or RNA that may be amplified by a polymerase chain reaction method, and may be, for example, total cell DNA, plasmid DNA, phage DNA, cDNA, or mRNA.

The reagent may be, for example, a buffer solution (suspension buffer) capable of suspending cells, a buffer solution (cell lysis buffer) helping dissolve cells, a primer required to amplify DNA and/or RNA by a polymerase chain reaction, a polymerase, deoxyribonucleotide triphosphate (dNTP), magnesium chloride (MgCl₂), a fluorophore which may be used as a reporter of the quantitative sensing of the amplified DNA and/or RNA, or a combination of at least two thereof. The fluorophore may include organic fluorophores, fluorescent protein, or quantum dots.

At least one chamber of the at least one chambers may include a plurality of nanostructures 200 fixed on the internal surface thereof, and the nanostructures 200 may have an independent form having a clear boundary with the surrounding medium.

The plurality of nanostructures 200 may be a nanoheater absorbing incident light from the light source and showing a photothermal phenomenon, a nanoantenna showing plasmon resonance absorption and a scattering phenomenon, or a combination thereof. The sample cell may be thermally lysed by heat generated by the photothermal phenomenon from the nanoheater, and the DNA or RNA may undergo a polymerase chain reaction to amplify the DNA or RNA. It may quantitatively sense DNA or RNA by the plasmon resonance absorption and the scattering phenomenon by the light incident on the nanoantenna.

A plurality of nanostructures 200 fixed on the surface of the at least one chamber may independently function as a nanoheater or a nanoantenna, and one nanostructure 200 may simultaneously function as both the nanoheater and the nanoantenna.

FIGS. 2 to 5 show a nanostructure 200 fixed on the internal surface of at least one chamber in the DNA and/or RNA detecting apparatus according to the present invention.

FIG. 2 is a cross-sectional side view of a spherically-shaped metal nanoparticle 230 according to an embodiment, and a′ refers to a radius of the spherically-shaped metal nanoparticle 230.

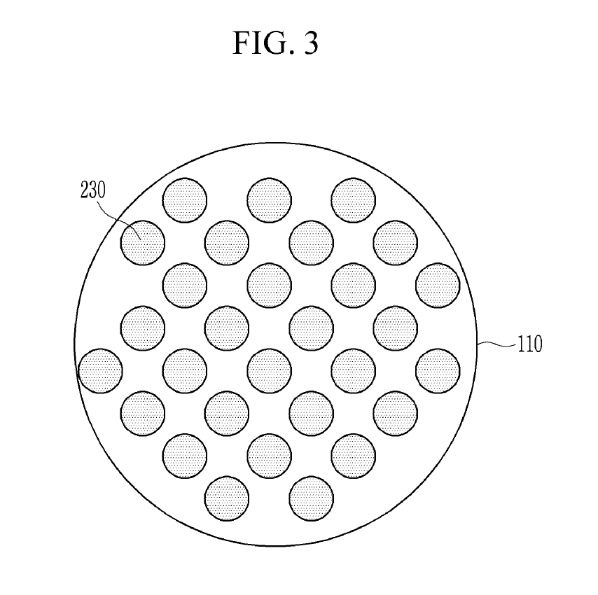

FIG. 3 is a schematic top plan view showing a form in which at least one of spherically-shaped metal nanoparticles 230 is fixed on the internal surface of at least one chamber formed on the substrate 100 in the detecting apparatus.

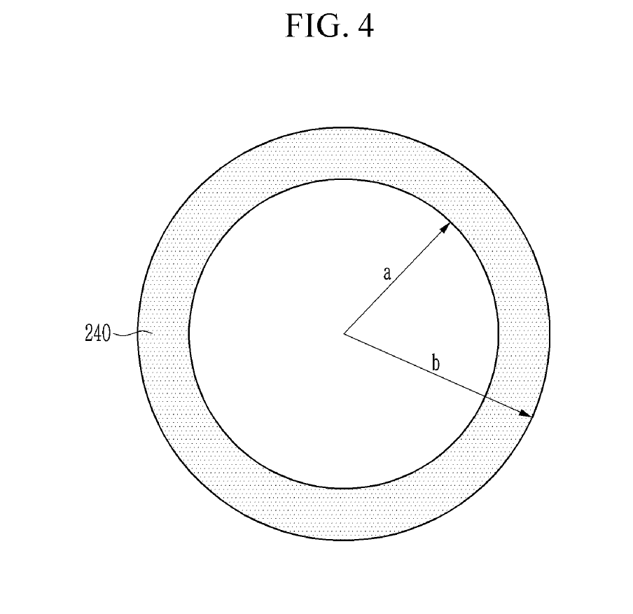

FIG. 4 is a cross-sectional side view of a core-shell-shaped nanoparticle 240 according to an embodiment, a refers to a radius of the core in the core-shell-shaped nanoparticle 240, and b refers to a radius of the entire nanoparticle including the core and the shell in the core-shell-shaped nanoparticle 240.

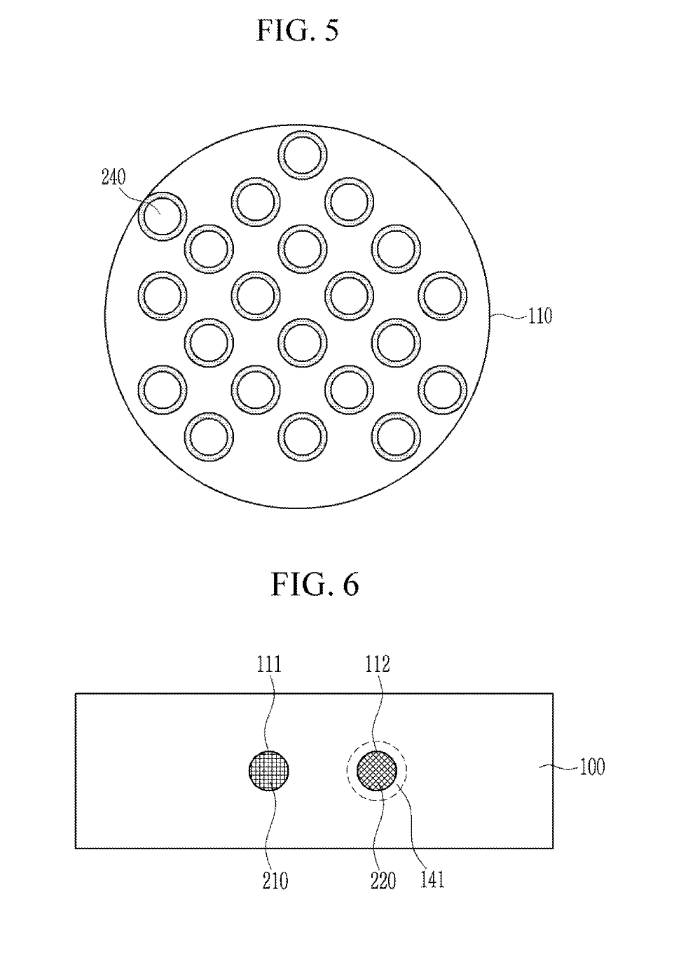

FIG. 5 is a schematic top plan view showing a form in which at least one of the core-shell-shaped nanoparticles 240 is fixed on the internal surface of at least one chamber formed on the substrate 100 in the detecting apparatus.

The nanostructure 200 may be a spherically-shaped metal nanoparticle 230 as shown in FIGS. 2 and 3, a nanoparticle 240 having a core-shell shape where the core is a dielectric and the shell is a metal as shown in FIGS. 4 and 5, or a combination thereof.

A plurality of spherically-shaped metal nanoparticles 230 fixed on the surface of at least one of the chambers may have excellent plasmon resonance absorption efficiency (σ_{abs}) of the irradiated light source. The plurality of core-shell-shaped nanoparticles 240 fixed on the surface of the at least one chamber may have a widely adjustable wavelength. Thus, a person of ordinary skill in the art may select a shape and an association of the nanostructure according to needs to realize a nanoantenna for easily sensing DNA and/or RNA.

The descriptions relating to the photothermal phenomenon, the plasmon resonance absorption efficiency (σ_{abs}), the heating power (σ_{abs}) of the nanostructures, and the temperature change (ΔT) of the surrounding medium which is changed by the nanostructure will be given later.

A metal in the spherically-shaped metal nanoparticle 230 may be one having a distinctive plasmon resonance effect in a wide wavelength region. The metal in the spherically-shaped metal nanoparticle may be gold (Au), silver (Ag), copper (Cu), aluminum (Al), or a combination thereof.

When using the metal, plasmon resonance absorption efficiency (o abs) of the spherically-shaped metal nanoparticle may be high in near infrared (NIR) region having a large thermal absorption cross-section of a body fluid, and cell thermal lysis and DNA or RNA denaturation may be easily performed. In addition, the plasmon resonance absorption and/or scattering efficiency is maximized in visible (VIS) region to easily perform quantitative sensing of DNA and/or RNA.

When a fluorophore is used as a reporter for the quantitative sensing of the amplified DNA and/or RNA, the reporter may be a commonly used fluorophore having a wide absorption wavelength from an ultraviolet (UV) region to a near infrared (NIR) region.

In the nanoparticle 240 having a core-shell shape where the core is a dielectric and the shell is a metal, the dielectric may include SiO₂, BK7 (made by Schott AG), SF10 (made by Schott AG), SF11 (made by Schott AG), N-LASF46A (made by Schott AG), a polystyrene-based polymer, a polymethylmethacrylate polymer, a polycarbonate-based polymer, a cyclic olefin copolymer, or a combination thereof, and the metal may include gold (Au), silver (Ag), copper (Cu), aluminum (Al), or a combination thereof. The core may be a dielectric having the same or a different material from the substrate 100.

As described above, a plurality of spherically-shaped metal nanoparticles 200 fixed on the surface of at least one chamber may have excellent plasmon resonance absorption efficiency (σ_{abs}) to the irradiated light source, and a plurality of core-shell-shaped nanoparticles 240 fixed on the surface of at least one chamber may have a widely adjustable wavelength of the plasmon resonance absorption, and a person of ordinary skill in the art may select an arbitrary combination of the shape and material of the nanostructures according to needs to realize a nanoantenna for easily performing the quantitative sensing of DNA and/or RNA.

The sizes of the nanostructures 200 may be about 1 nm to about 1000 nm, 5 nm to 500 nm, or 10 nm to 100 nm. When the nanostructure is a spherically-shaped metal nanoparticle 230, the size of the nanostructure means a particle diameter of the spherically-shaped nanoparticle. When the nanostructure is a core-shell-shaped nanoparticle 240, the size of the nanostructure means a particle diameter of the entire nanoparticle including the core and the shell. As will be described later, when the nanostructure 200 has the size in the range mentioned above, the nanostructure 200 may have excellent photothermal effects.

Although not shown in FIG. 1, the internal surface of at least one chamber may have a reflective coating. The chamber having the reflective coating may reflect the incident light to reduce loss of the light.

Although not shown in FIG. 1, the at least one chamber may include at least one thermo-cooler. The thermo-cooler may be disposed under the at least one chamber. A temperature of the chamber may be adjusted by controlling an on/off cycle of the thermo-cooler, and DNA or RNA may be amplified by adjusting a cycle of the polymerase chain reaction of the DNA or RNA and thermal lysis of the sample.

Although not shown in FIG. 1, the at least one light source may irradiate light having a wavelength in a range of about 10 nm to about 10 μm. The at least one light source may irradiate light having a different wavelength from the other light sources.

The at least one light source may independently irradiate light having a wavelength (hv₁) configured to increase a temperature of the surrounding medium by inducing a photothermal phenomenon of the nanostructures 200, light having a wavelength (hv₂) configured to perform plasmon resonance absorption of the nanostructures 200, maximization of scattering efficiency, or both of them, or light having wavelengths (hv₁ and hv₂) configured to perform plasmon resonance absorption of the nanostructures, maximization of scattering efficiency, or both of them, while increasing the temperature of the surrounding medium by inducing a photothermal phenomenon of the nanostructures.

In order for thermal lysis of cells and/or denaturation of DNA and/or RNA to occur easily, a person of ordinary skill in the art may select a wavelength (hv₁) configured to increase a temperature of the surrounding medium by inducing the photothermal phenomenon of the nanostructures 200, considering a wavelength for maximizing the plasmon resonance absorption of the nanostructure.

When a fluorophore is used as a reporter for the quantitative sensing of the amplified DNA and/or RNA, a person of ordinary skill in the art may select a wavelength (hv₂) configured to perform plasmon resonance absorption of the nanostructure, maximization of scattering efficiency, or both according to the needs, considering a wavelength for maximizing the absorption of the fluorophore.

The at least one light source may independently irradiate the at least one chamber with light.

The at least one light source may independently emit monochromatic light or polychromatic light, and desirably monochromatic light. For example, the monochromatic light may be laser light, for example, from a gas laser, a solid laser, a semiconductor laser, or a laser diode. The polychromatic light may be light emitted from a xenon lamp, a white LED, a light emitting diode, a halogen lamp, an infrared light source, and the like.

When the fluorophore is used as a reporter for sensing the amplified DNA and/or RNA, the monochromatic light may be light having a wavelength configured to maximize the absorption of the fluorophore, and the polychromatic light may be light having a wavelength range including a wavelength configured to maximize the absorption of the fluorophore.

Although not shown in FIG. 1, the apparatus according to the embodiment may further include at least one polarizer between the substrate 100 and the at least one light source, a color filter, or a combination thereof. The polarizer may control polarization of light having incident on the substrate 100. The color filter may select a wavelength of light incident on the substrate 100.

Even though not shown in FIG. 1, one or more light receiving units may be present on the same surface as the at least one light source with respect to the substrate 100, or may be present on opposite surfaces of the at least one light source in the center of the substrate 100.

When the fluorophore is used as a reporter for the quantitative sensing of the amplified DNA and/or RNA, the light-receiving unit may include at least one light detector capable of sensing a fluorescence signal of the fluorophore.

Although not shown in FIG. 1, the light-receiving unit may include a light detector capable of measuring the emitted and scattered optical signal caused by the plasmon resonance absorption and/or the scattering phenomenon of the nanostructure 200 fixed on the surface of the at least one chamber; and when the fluorophore is used as a reporter for sensing the amplified DNA and/or RNA, it may include a light detector capable of measuring a fluorescent signal of the fluorophore.

Even though not shown in FIG. 1, the apparatus according to the embodiment may further include at least one polarizer between the substrate 100 and the light-receiving unit, a color filter, or a combination thereof. The polarizer may control polarization of light reflected, transmitted, emitted, or scattered from the substrate 100. The color filter may select a wavelength of light that is reflected, transmitted, emitted, or scattered from a plurality of nanostructures 200, and for example, may select a wavelength of light that is reflected, transmitted, emitted, or scattered from a plurality of nanoantennas. When the fluorophore is used as a reporter, the color filter may be a one that is capable of selecting a wavelength of the fluorescent signal of the fluorophore.

Although not shown in FIG. 1, the detecting apparatus according to the embodiment may further include a control unit controlling an on/off cycle of the at least one light source and a wavelength of light incident on the at least one chamber. The detecting apparatus for at least one of DNA and RNA may further include a control unit controlling an on/off cycle of the thermo-cooler. The control unit controls thermal lysis of the sample, a cycle of the polymerase chain reaction of DNA and/or RNA by adjusting a wavelength of light incident on the at least one chamber, an on/off cycle of the at least one light source, and/or an on/off cycle of the thermo-cooler, so as to amplify DNA and/or RNA.

FIG. 6 is a schematic top plan view showing an apparatus according to another embodiment of the present invention in which a plurality of chambers are formed on one substrate.

In the detecting apparatus according to an embodiment shown in FIG. 6, the substrate 100 is the same as that shown in FIG. 1.

Referring to FIG. 6, in the detecting apparatus according to an embodiment, the at least one chamber may include at least one first heating chamber 111 and at least one sensing chamber 112.

The at least one first heating chamber 111 may support a sample, a reagent, or a combination thereof, and the sample and the reagent are the same as described above.

At least one first heating chamber 111 may include a plurality of nanostructures 210 fixed on the internal surface, and the nanostructures 210 are the same as the plurality of nanostructures 200 fixed on the internal surface of the at least one chamber shown in FIG. 1, and the same as described in FIGS. 2 to 5.

The plurality of nanostructures 210 fixed on the internal surface of at least one first heating chamber 111 may be the nanoheaters. The sample cell may be thermally lysed by heat generated from a photothermal phenomenon in a plurality of nanoheaters fixed on the internal surface of at least one first heating chamber 111.

The plurality of nanostructures 210 fixed on the internal surface of at least one first heating chamber 111 may be, for example, nanoparticles 240 having a core-shell shape where the core is a dielectric and the shell is a metal.

Although not shown in drawings, the internal surface of at least one first heating chamber 111 may have a reflective coating. The first heating chamber 111 having a reflective coating may reduce the light loss by reflecting the incident light.

In the at least one sensing chamber 112, DNA and/or RNA may be amplified by a polymerase chain reaction, and the amplified DNA and/or RNA may be sensed. The at least one sensing chamber 112 may include a plurality of nanostructures 220 fixed on the internal surface, and the nanostructures are the same as the plurality of nanostructures 200 fixed on the internal surface of the at least one chamber shown in FIG. 1, and the same as described in FIGS. 2 to 5.

The at least one sensing chamber 112 may include a plurality of nanostructures 220 fixed on the internal surface. For example, the plurality of nanostructures 220 fixed on the internal surface of the at least one sensing chamber 112 may be the nanoheater, the nanoantenna, the nanostructures capable of working both as a nanoheater and a nanoantenna simultaneously, or a combination thereof.

By periodically controlling a temperature change by the photothermal phenomenon of a plurality of nanoheaters fixed on the internal surface of the at least one sensing chamber 112, the polymerase chain reaction of DNA and/or RNA may be carried out, and DNA and/or RNA may be amplified in the at least one sensing chamber 112. Using the plasmon resonance absorption and the scattering phenomenon of light incident on the plurality of nanoantennas fixed on the internal surface of the at least one detecting chamber 112, it may sense the amplified DNA and/or RNA. The sensing may be quantitative sensing.

The plurality of nanostructures 220 fixed on the surface of at least one sensing chamber 112 may independently work as a nanoheater and/or a nanoantenna, and at least one of the nanostructures 220 may work as both a nanoheater and a nanoantenna.

The plurality of nanostructures 220 fixed on the surface of the at least one sensing chamber 112 may be nanoparticles 240 having a core-shell shape where the core is a dielectric and the shell is a metal.

At least one sensing chamber 112 may include at least one thermo-cooler 141. The thermo-cooler 141 is same as described above.

Even though not shown in the drawings, the apparatus according to an embodiment may include at least one light source, and the at least one light source is the same as described above.

The light source irradiating light having a wavelength (hv₁) configured to increase a temperature of the surrounding medium by inducing a photothermal phenomenon of the nanostructure may irradiate light to at least one first heating chamber 111 and/or at least one sensing chamber 112.

The light source irradiating light having a wavelength (hv₂) configured to perform plasmon resonance absorption of the nanostructure, maximization of the scattering efficiency, or both may irradiate light to at least one sensing chamber 112.

The light source irradiating light having a wavelength configured to perform plasmon resonance absorption of the nanostructure, maximization of scattering efficiency, or both while increasing a temperature of the surrounding medium by inducing a photothermal phenomenon of the nanostructures may irradiate light to at least one first heating chamber 111 and/or at least one sensing chamber 112.

Although not shown in FIG. 6, the detecting apparatus according to the embodiment may further include the polarizer, the color filter, the light-receiving unit, and the control unit.

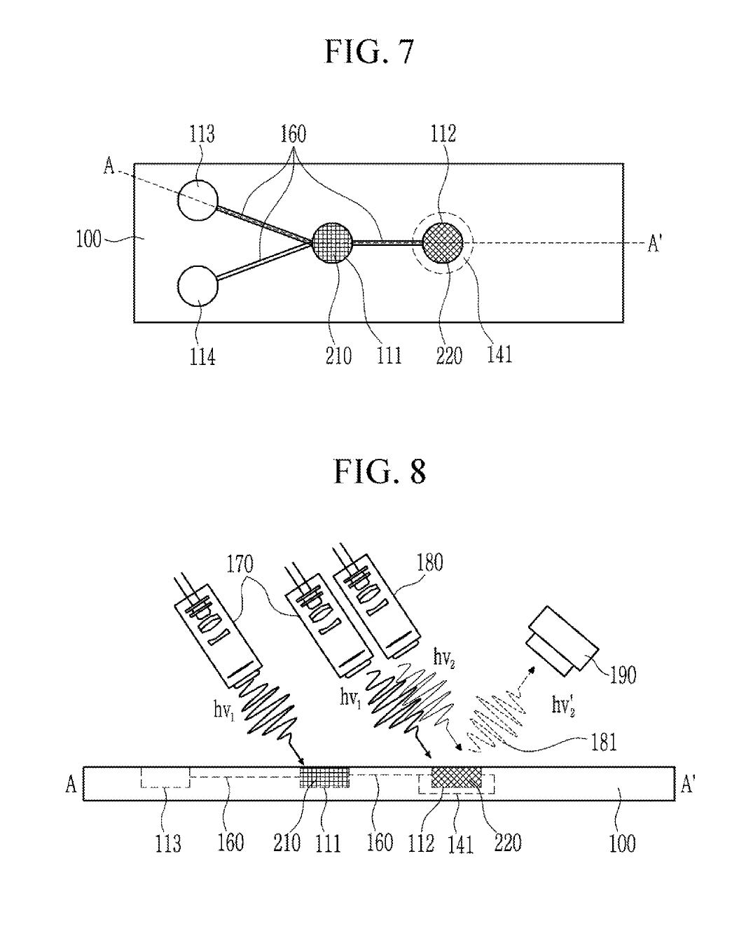

FIG. 7 is a schematic top plan view of an apparatus including a plurality of chambers and at least one fluid channel formed on a substrate according to an embodiment.

In the apparatus according to an embodiment shown in FIG. 7, a substrate 100, at least one first heating chamber 111, at least one sensing chamber 112, a plurality of nanostructures 210 fixed on the internal surface of the at least one first heating chamber 111, a plurality of nanostructures 220 fixed on the internal surface of the at least one sensing chamber 112, and a thermo-cooler 141 are the same as illustrated in FIGS. 1 to 6.

Referring to FIG. 7, in the apparatus according to an embodiment, the at least one chamber may further include at least one sample chamber 113, and may further include at least one fluid channel 160 connecting the at least one sample chamber 113 with the at least one first heating chamber 111, and at least one fluid channel 160 connecting the at least one first heating chamber 111 with the at least one sensing chamber 112. The at least one fluid channel 160 may independently include at least one filter.

At least one sample chamber 113 may support the sample or a mixture of the sample and the reagent. The sample or the mixture of the sample and the reagent may be transported from at least one sample chamber 113 to at least one first heating chamber 111 through the fluid channel 160 connecting the at least one sample chamber 113 with the at least one first heating chamber 111. The sample which is thermally lysed in the first heating chamber 111 or the mixture of the sample and the reagent may be transported from at least one first heating chamber 111 to at least one sensing chamber 112 through the fluid channel 160 connecting the at least one first heating chamber 111 with the at least one sensing chamber 112.

The at least one chamber may further include at least one reagent chamber 114 and at least one fluid channel 160 connecting the at least one reagent chamber 114 with the at least one first heating chamber 111. The at least one fluid channel 160 may independently include at least one filter.

At least one reagent chamber 114 may support the reagent or a mixture of the sample and the reagent. The reagent or the mixture of the sample and the reagent may be transported from at least one reagent chamber 114 to at least one first heating chamber 111 through the fluid channel 160 connecting the at least one reagent chamber 114 with the at least one first heating chamber 111.

Although not shown in FIG. 7, the at least one chamber may further include at least one waste chamber, and may additionally include at least one fluid channel connecting the at least one waste chamber with at least one sensing chamber 112. The at least one fluid channel may independently include at least one filter. The at least one waste chamber may support a material to be removed while amplifying DNA and/or RNA in at least one sensing chamber 112, a material to be removed while sensing the amplified DNA and/or RNA, waste, or a combination thereof.

Although not shown in FIG. 7, the detecting apparatus shown in FIG. 7 may further include at least one light source, a polarizer, a color filter, a light-receiving unit, and a control unit described with respect to FIGS. 1 and 6, and each of the components is the same as described above.

FIG. 8 is a schematic cross-sectional side view showing an apparatus further including light sources 170 and 180 and a light-receiving unit 190 disposed on the same surface as the light sources 170 and 180 together in a cross-sectional view cut along a line A-A′ of the substrate shown in FIG. 7. A sample is put into at least one chamber in the apparatus, and light is irradiated from the light source 180 thereto, and then an optical signal 181 emitted from the chamber may be sensed by the light-receiving unit 190.

In the apparatus shown in FIG. 8, the substrate 100, at least one first heating chamber 111, at least one sensing chamber 112, a plurality of nanostructures 210 fixed on the internal surface of the at least one first heating chamber 111, a plurality of nanostructures 220 fixed on the internal surface of the at least one sensing chamber 112, a thermo-cooler 141, at least one sample chamber 113, and a fluid channel 160 are all the same as described for FIGS. 1 to 7.

In FIG. 8, the light source 170 may emit light having a wavelength range (hv₁) configured to increase a temperature of the surrounding medium by inducing a photothermal phenomenon of the nanostructures to at least one first heating chamber 111 and/or at least one sensing chamber 112.

The light source 180 may emit light having a wavelength range (hv₂) configured to perform plasmon resonance absorption of the nanostructures, maximization of the scattering efficiency, or both to at least one sensing chamber 112.

Absorbing or scattering due to plasmon resonance absorption of the nanostructures 220 fixed on the internal surface of the detecting chamber and/or a scattering phenomenon by the incident light from the light source 180 to the detecting chamber 112 may occur, and the optical signal 181 having a wavelength (hv₂′) emitted or scattered from the nanostructures 220 may be sensed by the light-receiving unit 190.

Although not shown in FIG. 8, the detecting apparatus according to an embodiment may further include at least one of the polarizer, the color filter, and the control unit, and the descriptions relating to each of them are the same as described above.

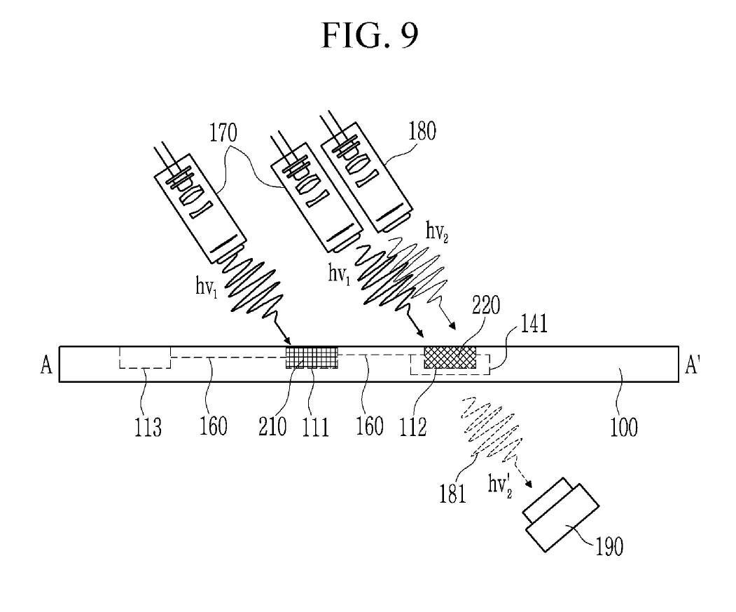

FIG. 9 is a schematic cross-sectional side view showing an apparatus further including light sources 170 and 180 and a light-receiving unit 190 disposed on opposite surfaces of the light sources 170 and 180 in the center of the substrate, together with the cross-sectional side view cut along the line A-A′ of the substrate shown in FIG. 7. As shown in FIG. 8, a sample is put into at least one chamber in the apparatus, light is irradiated from the light source 180 thereto, and an optical signal 181 emitted from the chamber may be sensed by the light-receiving unit 190.

In the apparatus according to an embodiment shown in FIG. 9, a substrate 100, at least one first heating chamber 111, at least one sensing chamber 112, a plurality of nanostructures 210 fixed on the internal surface of the at least one first heating chamber 111, a plurality of nanostructures 220 fixed on the internal surface of the at least one sensing chamber 112, a thermo-cooler 141, at least one sample chamber 113, a fluid channel 160, at least one light source 170 and 180, and a light-receiving unit 190 are the same as illustrated in FIGS. 1 to 8. In addition, although not shown, the apparatus according to an embodiment may further include at least one of a polarizer, a color filter, and a control unit, and each component of them is the same as described above.

Hereinafter, the effects of the nanostructure's shape and size on the photothermal phenomenon of the nanostructures, the plasmon resonance absorption efficiency (σ_{abs}), the heating power (Q_{abs}), the temperature change (ΔT) of the surrounding medium caused by the nanostructures, and the like are described.

The photothermal phenomenon of the nanostructures is proportional to the plasmon resonance absorption efficiency (σ_{abs}) in which the external light having a predetermined wavelength is absorbed in the nanostructures. The plasmon resonance absorption efficiency (σ_{abs}) is determined by a dielectric property of the material for the nanostructures, a nanostructure shape, and a nanostructure size, which may be calculated by the following Equation 1.

$${σ}_{abs} = \frac{k}{{ɛ}_{0} {\vert {\vec{E}}_{0} \vert}^{2}} {\int }_{v} 𝕀 m {ɛ}_{m} d \vec{r} {\vert \vec{E} ( {r}^{->} ) \vert}^{2} \qquad [ Equation 1 ]$$

In Equation 1,

k is a wave vector of the external light (for example, a wave vector of the external electric field), εₘ is a dielectric function of the nanostructures, co is a dielectric function of the surrounding medium, E₀ is the external electric field in the case of without nanostructures, E is the sum of electric fields E₀ and the electric field induced in nanostructures by charge polarization, r is a position vector from a center (origin) to an arbitrary point located in space, and ∫ᵥ is a volume integral performed over the surface of the nanostructures

Accordingly, a person having ordinary skill in the art may easily select or design a nanostructure to provide a photothermal phenomenon within a range suitable for the purpose and the usages for the nanostructures by adjusting the plasmon resonance absorption efficiency (σ_{abs}) of the nanostructures represented by Equation 1.

The heating power (Q_{abs}) of the nanostructures configured to increase a temperature of the surrounding medium is proportional to an intensity of the externally irradiated light. The heating power (Q_{abs}) at a steady state of the nanostructures may be calculated by the following Equation 2, and the temperature change (ΔT) of the surrounding medium changed by the nanostructures may be calculated by the following Equation 3.

$${Q}_{abs} = {σ}_{abs} I = {σ}_{abs} \frac{nc {ɛ}_{0}}{2} {\vert {\vec{E}}_{0} \vert}^{2} \qquad [ Equation 2 ]$$

In Equation 2,

σ_{abs} denotes plasmon resonance absorption efficiency of the nanostructure, I denotes intensity of irradiated light, n denotes a refractive index of the surrounding medium, c denotes a light velocity, ε₀ denotes a dielectric function of the surrounding medium, and E₀ is an electric field of the externally irradiated light.

$$Δ T = \frac{{Q}_{abs}}{4 π R κ} = \frac{{σ}_{abs} I}{4 π R κ} \qquad [ Equation 3 ]$$

In Equation 3,

Q_{abs} denotes a heating power of the nanostructures in a steady state, R denotes a radius of the nanostructures, and K denotes thermal conductivity of the surrounding medium.

Accordingly, a person of ordinary skill in the art may easily calculate the heating power at the steady state of the nanostructures to be used and the temperature change (ΔT) of the surrounding medium changed by the nanostructures, from Equations 2 and 3, and thereby may select or design nanostructures suitable for the purpose and the usages thereof.

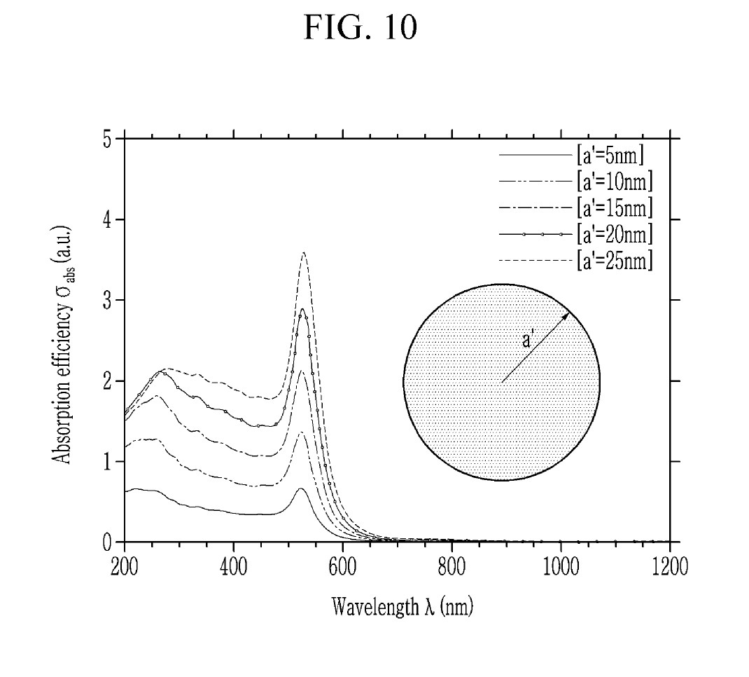

FIG. 10 is a graph showing plasmon resonance absorption efficiency (σ_{abs}) of spherically-shaped gold nanoparticles in water, which is changed depending upon a radius (a′) of the spherically-shaped gold nanoparticles.

Referring to FIG. 10, a wavelength of the spherically-shaped metal nanoparticles 230 having the maximum plasmon resonance absorption efficiency (σ_{abs}) is shifted to a side of long wavelength (red shift) according to an increasing radius (a′) of the spherically-shaped metal nanoparticle 230, but the magnitude of wavelength change is minimal.

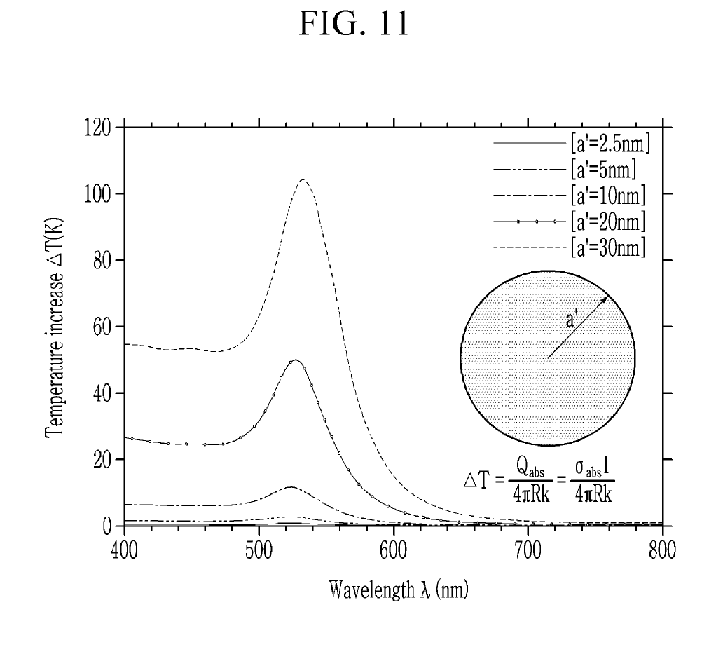

FIG. 11 is a graph showing a temperature change (ΔT) of water according to plasmon resonance absorption of the spherically-shaped gold nanoparticles in water, which is changed depending upon a radius (a′) of the spherically-shaped gold nanoparticles.

Referring to FIG. 11, the temperature change of water, which is the surrounding medium, is about ΔT≈105 K when the spherically-shaped gold nanoparticles having a radius of about 30 nm in water having thermal conductivity of 0.58 W/mK are irradiated with a light having an intensity of about I=2 mW/pmt in a wavelength of λ=550 nm. The temperature change is a change of a temperature capable of sufficiently lysing cells and/or a temperature capable of amplifying DNA and/or RNA by a polymerase chain reaction when irradiating light having a wavelength range (hv₁) configured to increase a temperature of the surround medium by inducing a photothermal phenomenon of the nanostructures in the first heating chamber 111 or the detecting chamber 112. Referring to FIGS. 10 and 11, when a plurality of nanostructures 200 fixed on the internal surface of the at least one chamber 110 are the spherically-shaped metal nanoparticles 230, it may generate a temperature change to provide excellent plasmon resonance absorption efficiency (σ_{abs}) to thermally lyse cells, and/or to amplify DNA and/or RNA by the polymerase chain reaction.

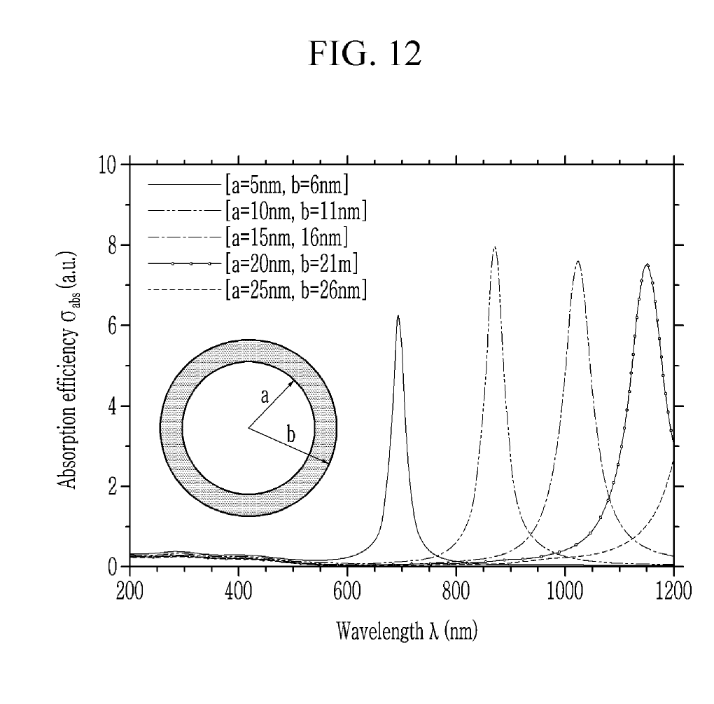

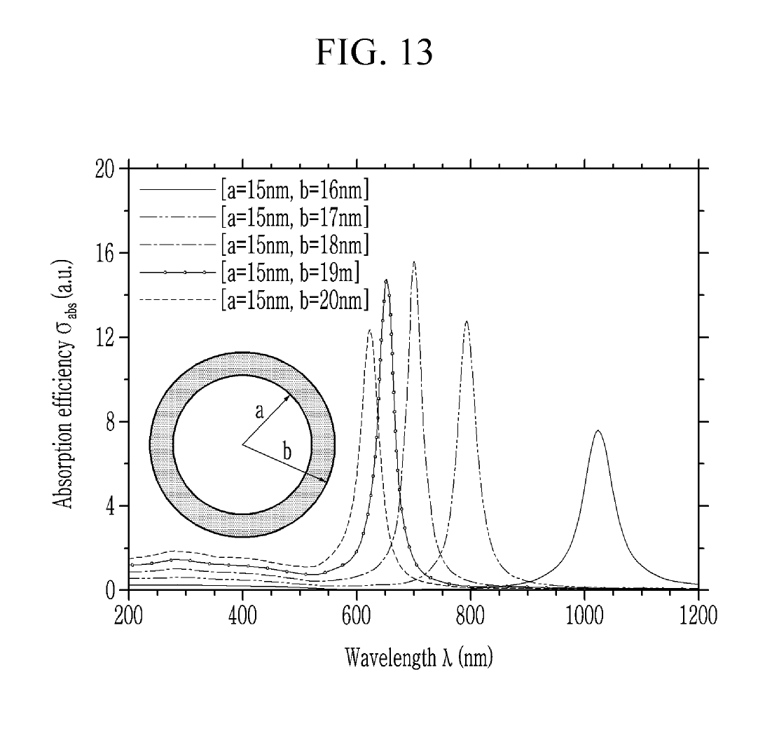

FIG. 12 shows the results of theoretically calculating plasmon resonance absorption efficiency (σ_{abs}) of the core-shell-shaped nanoparticles in water, which is changed depending upon a radius (a) of a silica core, in the nanoparticles having a core-shell shape where the core is silica and the shell is gold having a thickness of about 1 nm.

Referring to FIG. 12, the wavelength configured to maximize plasmon resonance absorption efficiency (σ_{abs}) is significantly changed according to the radius (a) of the core in the core-shell-shaped nanoparticles 240. In addition, when the radius (a) of the core increases while maintaining the thickness of the shell, the wavelength configured to maximize the plasmon resonance absorption efficiency (σ_{abs}) shifts to a longer wavelength side (red shift).

FIG. 13 shows results of theoretically calculating plasmon resonance absorption efficiency (σ_{abs}) of the core-shell-shaped nanoparticles in water, which is changed depending upon a thickness of the shell, in the nanoparticles having a core-shell shape where the core is silica having a radius of about 15 nm (a) and the shell is gold (Au).

Referring to FIG. 13, the wavelength configured to maximize the plasmon resonance absorption efficiency significantly changes according to the thickness of the shell (b-a) of the core-shell-shaped nanoparticles 240. In addition, when the thickness of the shell (b-a) increases while maintaining the radius (a) of the core, the wavelength configured to maximize the plasmon resonance absorption efficiency (σ_{abs}) shifts to a shorter wavelength side (blue shift).

Referring to FIGS. 12 and 13, the nanostructures 200 fixed on the internal part of the at least one chamber 110 may satisfy a condition that the nanostructures have the maximum absorption efficiency to the light having a wavelength (hv₁) configured to increase a temperature of the surrounding medium by inducing the photothermal phenomenon of the nanostructures, which is the condition where the photothermal effect is maximized by the absorbed light. The nanostructures satisfying the condition may function as the nanoheaters.

When the nanostructures 200 fixed in the at least one chamber 110 are nanoparticles 240 having a core-shell shape where the core is a dielectric and the shell is a metal, a person of ordinary skill in the art may realize nanoheaters having a wide wavelength range configured to maximize adjustable plasmon resonance absorption efficiency (σ_{abs}) and may easily change the wavelength range configured to maximize absorption efficiency (σ_{abs}) according to plasmon resonance even by slightly changing a thickness (b-a) of the shell and/or a radius (a) of the core, so as to provide nanoheaters configured to easily maximize heating efficiency by the photothermal phenomenon.

In addition, when the nanostructures 200 fixed in the at least one chamber 110 are nanoparticles 240 having a core-shell shape where the core is a dielectric and the shell is a metal, an adjustable wavelength range having maximum plasmon resonance absorption efficiency (σ_{abs}) may be wide. Therefore, a person of ordinary skill in the art may select an arbitrary combination of the shape and material of the nanostructures to realize a nanoantenna for easily performing the quantitative sensing of DNA and/or RNA.

Accordingly, the detecting apparatus according to an embodiment may be realized as a biochip or a biosensor sensing at least one of DNA and RNA. For example, the sensing may be quantitative sensing.

Another embodiment provides a detecting apparatus for at least one of DNA and RNA according to an embodiment and a kit for sensing at least one of DNA and RNA including a sample including at least one of DNA and RNA and a reagent that is reactive with the sample.

The sample including at least one of DNA and RNA and the reagent are the same as described above, so the detailed descriptions are omitted.

The reagent may be supported in the at least one chamber in the detecting apparatus for at least one of DNA and RNA, or may be present separately from the detecting apparatus for at least one of DNA and RNA.

Hereinafter, a method of sensing at least one of DNA and RNA using the detecting apparatus for at least one of DNA and RNA according to an embodiment will be described.

A sensing method of at least one of DNA and RNA according to an embodiment includes: adding a sample including one or more of DNA and RNA to be analyzed, a polymerase, and a base to at least one chamber formed on a substrate of the detecting apparatus according to the embodiment and having a plurality of nanostructures fixed on an inner surface thereof; irradiating the chamber with light to adjust the temperature of the sample and to perform a polymerization reaction so that at least one of DNA and RNA including a specific sequence in at least one of the DNA and RNA is amplified; and irradiating the chamber in which the polymerization reaction is completed with light to sense an absorbed or scattered optical signal.

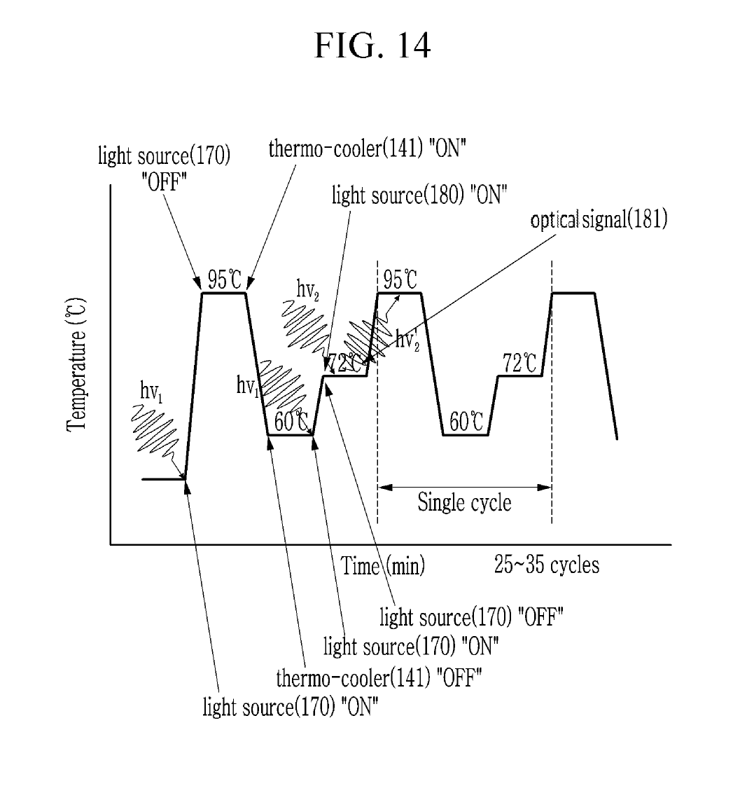

FIG. 14 schematically shows a method of changing a temperature by adjusting an operation time of a light source and a thermo-cooler according to time, in order to amplify and quantitatively sense at least one of DNA and RNA in the at least one chamber.

Referring to FIG. 14, in the method of quantitatively sensing at least one of DNA and RNA according to an embodiment, the inner temperature of at least one chamber may be heated up to about 95° C. by irradiating the nanostructures fixed on the internal surface of at least one chamber with light having a wavelength (hv₁) configured to increase a temperature of the surrounding medium by inducing a photothermal phenomenon of the nanostructures, thus at least one of DNA and RNA in the at least one chamber may be denatured into a single strain.

Then the light having a wavelength (hv₁) configured to increase a temperature of the surrounding medium by inducing a photothermal phenomenon of the nanostructure is turned off, and a thermo-cooler 141 is turned on to cool the inner part of the sensing chamber, thus the single strand of the denatured DNA and/or RNA may be annealed with a primer (primer annealing). The primer has a base sequence that is complementary to the substrate sequence of the denatured DNA and/or RNA single strand, and the cooled temperature (Tm) may be determined by the number of G base (guanine), C base (cytosine), A base (adenine), and T base (thymine) included in the primer. The cooled temperature may be calculated by the following Equation 4. When the temperature is cooled to the temperature calculated by the following Equation 4, the thermo-cooler 141 may be turned off.

Tm(° C.)=(4×[G+C])+(2×[A+T]) [Equation 4]

In Equation 4,

G is the number of guanine bases included in the primer, C is the number of cytosine bases included in the primer, A is the number of adenine bases included in the primer, and T is the number of thymine bases included in the primer.

Thereafter, it is irradiated with light having a wavelength (hv₁) configured to increase a temperature of the surrounding medium from the light source 170 to the chamber by inducing a photothermal phenomenon of the nanostructures, so that the inner part of the chamber is heated up to an activation temperature of DNA polymerase.

A dNTP base that is complementary to the DNA and/or RNA single strand is sequentially polymerized at the 3′ terminal end of the primer annealed to the DNA and/or RNA single strand by activating the DNA polymerase, so as to polymerize a DNA double strand.

The DNA polymerase may be, for example, a Taq polymerase, and in the case of the Taq polymerase, the activation temperature may be about 72° C. A maintaining time of the activation temperature of the polymerase in the chamber may be appropriately selected according to a length of a specific sequence to be amplified in the DNA and/or RNA and a polymerization rate of the DNA polymerase.

The wavelength (hv₁) capable of increasing a temperature of the surrounding medium by inducing a photothermal phenomenon of nanostructures, an on/off cycle of the light source, and an on/off cycle of the thermo-cooler 141 may be controlled to adjust a maintaining time of the activation temperature of the polymerase.

One cycle may consist of: denaturing the DNA and/or RNA to a single strand (Denaturation); annealing the denatured DNA and/or RNA single strand and a primer (Primer Annealing); and sequentially polymerizing the dNTP base that is complementary to the DNA and/or RNA single strand at a 3′ terminal end of the primer annealed on the DNA and/or RNA single strand by the DNA polymerase to polymerize a DNA double strand (Polymerization), and the one cycle may be repeated several times.

At least one chamber including a plurality of nanostructures fixed on an inner surface thereof may be irradiated with light (hv₂) having a wavelength range configured to perform plasmon resonance absorption of the nanostructures, maximization of scattering efficiency, or both of them, and the irradiated light may be absorbed by plasmon resonance absorption of the nanostructures and/or a scattering phenomenon, and an emitted or scattered wavelength (hv₂′) may be sensed by the light-receiving unit 190 to sense amplified DNA. The sensing may be quantitative sensing.

Every one cycle or every plurality of cycles, it is irradiated from the light source 180 to at least one chamber including a plurality of nanostructures fixed on an internal surface thereof with light having a wavelength (hv₂) configured to perform plasmon resonance absorption of the nanostructures, maximization of scattering efficiency, or both of them; and a light-receiving unit 190 senses a reflected or transmitted optical signal of remaining light after the plasmon resonance absorption of the nanostructures or after the scattering phenomenon absorption and/or an emitted or scattered optical signal 181 from there, so as to detect the amplified DNA every one cycle or every plurality of cycles. The sensing may be quantitative sensing.

Although not shown in drawings, when the sample is a cell, a cell suspension, or a nucleus of the cell, before polymerizing at least one of DNA and RNA including a specific sequence in the at least one of DNA and RNA, it may further include performing thermal lysis of the sample with heat generated from the photothermal phenomenon of the plurality of nanostructures 200, by irradiating light to at least one chamber including a plurality of nanostructures fixed on the internal surface thereof and formed on the substrate to adjust a temperature of the sample. A method of sensing DNA and/or RNA present therein after the thermal lysis of the sample is the same as above.

While this disclosure has been described in connection with what is presently considered to be practical example embodiments, it is to be understood that the invention is not limited to the disclosed embodiments, but, on the contrary, is intended to cover various modifications and equivalent arrangements included within the spirit and scope of the appended claims.

### DESCRIPTION OF SYMBOLS

100: substrate
110: one chamber
111: first heating chamber
112: sensing chamber
113: sample chamber
114: reagent chamber
141: thermo-cooler
160: fluid channel
170, 180: light source
181: optical signal emitted or scattered after the plasmon resonance absorption of the nanostructures
190: light-receiving unit
200, 210, 220: nanostructures
230: spherically-shaped metal nanoparticle
240: nanoparticle having a core-shell shape where the core is a dielectric and the shell is a metal
a: radius of core of nanoparticle having a core-shell shape
b: radius including the core and the shell of entire nanoparticle having a core-shell shape
a′: radius of spherically-shaped nanoparticle

What is claimed is:

1. A detecting apparatus for at least one of DNA and RNA, comprising:
a substrate;
at least one chamber formed on the substrate;
a plurality of nanostructures fixed on the internal surface of the at least one chamber;
at least one light source configured to supply incident light on the at least one chamber; and
a light-receiving unit configured to sense an optical signal from the nanostructures.

2. The detecting apparatus of claim 1, wherein the at least one chamber comprises at least one first heating chamber and at least one sensing chamber.

3. The detecting apparatus of claim 2, wherein
the at least one chamber further comprises at least one sample chamber,
at least one fluid channel connecting the at least one sample chamber with the at least one first heating chamber, and
at least one fluid channel connecting the at least one first heating chamber with the at least one sensing chamber.

4. The detecting apparatus of claim 3, wherein
the at least one chamber further comprises at least one reagent chamber, and
at least one fluid channel connecting the at least one reagent chamber with the at least one first heating chamber.

5. The detecting apparatus of claim 1, wherein the substrate is transparent glass or a polymer having a refractive index of about 1.3 to about 1.9.

6. The detecting apparatus of claim 5, wherein the glass comprises one of SiO₂, BK7, SF10, SF11, N-LASF46A, and a combination thereof, and
the polymer comprises a polystyrene-based polymer, a polymethylmethacrylate polymer, a polycarbonate-based polymer, a cyclic olefin copolymer, or a combination thereof.

7. The detecting apparatus of claim 1, wherein the nanostructures are spherically-shaped metal nanoparticles,
nanoparticles having a core-shell shape where the core is a dielectric and the shell is a metal, or
a combination thereof.

8. The detecting apparatus of claim 7, wherein, in the spherically-shaped metal nanoparticles, the metal is gold (Au), silver (Ag), copper (Cu), aluminum (Al), or a combination thereof.

9. The detecting apparatus of claim 7, wherein
in the nanoparticles having a core-shell shape where the core is a dielectric and the shell is a metal,
the dielectric comprises SiO₂, BK7, SF10, SF11, N-LASF46A, a polystyrene-based polymer, a polymethylmethacrylate polymer, a polycarbonate-based polymer, a cyclic olefin copolymer, or a combination thereof, and
the metal comprises gold (Au), silver (Ag), copper (Cu), aluminum (Al), or a combination thereof.

10. The detecting apparatus of claim 7, wherein a size of the nanostructures is about 1 nm to about 1000 nm.

11. The detecting apparatus of claim 2, wherein an internal surface of the at least one first heating chamber has a reflective coating.

12. The detecting apparatus of claim 2, wherein the at least one sensing chamber comprises at least one thermo-cooler.

13. The detecting apparatus of claim 1, wherein the at least one light source irradiates light in a wavelength range of about 10 nm to about 10 μm.

14. The detecting apparatus of claim 1, wherein the at least one light source independently irradiates
light having a wavelength range configured to increase a temperature of the surrounding medium by inducing a photothermal phenomenon of the nanostructures,
light having a wavelength range configured to perform plasmon resonance absorption of the nanostructures, maximization of scattering efficiency, or both, or
light having a wavelength range configured to perform plasmon resonance absorption of the nanostructures by inducing a photothermal phenomenon of the nanostructures and to maximize scattering efficiency, or both, while increasing the temperature of the surrounding medium.

15. The detecting apparatus of claim 1, wherein the at least one light source independently emits monochromatic light or polychromatic light.

16. The detecting apparatus of claim 1, which further comprises at least one of a polarizer, a color filter, or a combination thereof between the substrate and the at least one light source.

17. The detecting apparatus of claim 1, wherein one or more light receiving units are present on the same surface as the at least one light source with respect to the substrate, or
are present on opposite surfaces of the at least one light source in the center of the substrate.

18. The detecting apparatus of claim 1, which further comprises a control unit configured to control a wavelength of incident light on the at least one chamber and an on/off cycle of the at least one light source.

19. A kit comprising
the detecting apparatus for at least one of DNA and RNA of claim 1, and
a reagent to react with a sample comprising at least one of DNA and RNA.

20. A sensing method of at least one of DNA and RNA comprising a specific sequence, comprising:
adding a sample including one or more of DNA and RNA to be analyzed, a polymerase, and a base to at least one chamber formed on a substrate and having a plurality of nanostructures fixed on an inner surface thereof of the detecting apparatus for at least one of DNA and RNA of claim 1;
irradiating the chamber with light to adjust the temperature of the sample and to perform a polymerization reaction so that at least one of DNA and RNA including a specific sequence in at least one of the DNA and RNA is amplified; and
irradiating the chamber in which the polymerization reaction is completed with light to sense an absorbed or scattered optical signal.
